(19) United States
(12) Patent Application Publication
(10) Pub. No.: US 2026/0088170 A1
(43) Pub. Date: Mar. 26, 2026

(54) RADIOMICS BASED METHOD FOR PREDICTING THE ONSET OF HUMAN DISEASES USING NEURAL NETWORKS AND COLOR SPACE ANALYSIS

(71) Applicant: SHUBHAM CHANDRA, Katy, TX (US)

(72) Inventor: SHUBHAM CHANDRA, Katy, TX (US)

(21) Appl. No.: 18/895,105

(22) Filed: Sep. 24, 2024

(51) Int. Cl. G16H 50/20; A61B 6/03; A61B 6/50; G06T 3/4046; G06T 5/60; G06T 5/94; G06T 7/00; G06T 7/11; G06T 7/90; G06T 11/00; G06T 11/20; G06V 10/25; G06V 10/764; G06V 10/82

(52) U.S. Cl. CPC G16H 50/20; G06T 3/4046; G06T 5/60; G06T 5/94; G06T 7/0014; G06T 7/11; G06T 7/90; G06T 11/10; G06T 11/26; G06V 10/25; G06V 10/764; G06V 10/82; A61B 6/032; A61B 6/501; G06T 2207/10024; G06T 2207/20076; G06T 2207/20081; G06T 2207/20084; G06T 2207/20132; G06T 2207/30016; G06T 2207/30096; G06T 2210/41; G06V 2201/031

(57) ABSTRACT

The present invention provides a radiomics-based method and system for predicting the onset of human diseases using medical imaging and advanced machine learning techniques. This non-invasive approach combines Convolutional Neural Networks (CNNs) with pseudo-color transformation in the CIELAB color space to enhance early disease detection. The method begins by acquiring grayscale medical images from diagnostic techniques such as CT, MRI, or X-ray, followed by CNN-based feature extraction to identify clinically relevant regions of interest. These regions are then converted into pseudo-color representations using the CIELAB color space, improving tissue contrast and visualization of subtle abnormalities. A machine learning classifier is applied to the pseudo-colored images to predict the likelihood of disease onset, generating an output report that includes a heatmap, probability score, and diagnostic recommendations. The invention offers a fully automated process that facilitates early detection, improved visualization, and personalized diagnostics, providing a versatile solution for various medical conditions.

### BACKGROUND

### Technical Field

The present invention relates to medical diagnostics using advanced radiomics and machine learning techniques. More specifically, it involves a non-invasive method for predicting the onset of human diseases by analyzing diagnostic imaging using a combination of convolutional neural networks (CNNs) and pseudo-color transformation based on the CIELAB color space. In lieu of this patent application and research, no federally/Government sponsored research or development-based funding was used.

### TECHNICAL BACKGROUND OF THE INVENTION

Today, medical imaging techniques such as CT scans play a vital role in diagnosing diseases by providing detailed visualizations of the internal body. However, early detection of disease remains a challenge, as radiologists often rely on manual image interpretation, which can be subjective and prone to error. Subtle changes in tissue structure may go unnoticed, delaying the diagnosis of conditions like cancer and cardiovascular diseases.

Recent advances in artificial intelligence (AI), particularly CNNs, and radiomics have enabled more precise analysis of medical images. Radiomics involves extracting large amounts of quantitative features from medical images that go beyond human visual perception. These radiomic features can uncover subtle image patterns, aiding in early disease detection.

Several approaches are currently being utilized for radiomics and human disease detection, spanning from traditional image processing techniques to advanced machine learning and artificial intelligence (AI) models. These methods leverage medical imaging data to extract meaningful features, helping in the diagnosis and prediction of disease outcomes. Traditional image processing and feature extraction represent one of the earliest approaches, where predefined algorithms and statistical methods are employed to extract specific features from medical images such as CT, MRI, or PET scans. These features include geometric properties like tumor size, volume, texture patterns, pixel intensity, wavelet transformation for texture analysis, and first-order statistics like mean and variance. These extracted features are typically used alongside statistical models to classify or predict disease characteristics such as tumor malignancy or prognosis.

Deep learning-based approaches, particularly Convolutional Neural Networks (CNNs), have gained traction due to their ability to automatically learn and extract complex features from medical images without manual intervention. CNNs, which excel at capturing spatial hierarchies in data, can be trained on large datasets to identify intricate patterns in medical images that correlate with disease outcomes. For medical imaging, 3D CNNs are often used because they handle volumetric data (e.g., CT or MRI scans) better than traditional 2D CNNs. Additionally, Recurrent Neural Networks (RNNs) and Long Short-Term Memory (LSTM) networks are sometimes combined with CNNs to process sequential data, such as tracking tumor changes over time.

Hybrid approaches, which combine traditional handcrafted feature extraction with deep learning models, aim to improve the performance of radiomics. Manually extracted radiomic features are integrated with deep learning models like CNNs, allowing these hybrid systems to capture both low-level and high-level representations of the data. Another hybrid approach integrates radiomic features with clinical data (e.g., patient age, sex, genetic markers), creating multi-modal models that better predict disease outcomes, especially in fields like radiogenomics where genomic data is included.

Radiomics-based machine learning models such as Support Vector Machines (SVM), Random Forests, k-Nearest Neighbors (k-NN), and logistic regression are also widely used. These models classify disease states based on the extracted radiomic features. Transfer learning, a method where a deep learning model is pre-trained on a large general dataset and fine-tuned for medical imaging, is particularly useful when there is limited labeled medical data. This technique has been adopted in radiomics for detecting rare diseases or for smaller datasets.

Radiomics is increasingly being integrated with multi-omics data (genomics, proteomics, transcriptomics) in a practice known as radiogenomics, which seeks to link imaging data with genetic and molecular insights. This has been particularly effective in oncology for predicting treatment responses. Additionally, natural language processing (NLP) is sometimes combined with radiomics to correlate radiology reports with image-based features, creating hybrid models that blend textual and visual information for better disease prediction.

Time-series analysis in radiomics helps track the evolution of diseases through sequential scans, with RNNs and LSTMs often applied to detect changes over time, particularly for tracking tumor growth. In cases where labeled data is scarce, unsupervised learning techniques such as k-means clustering and autoencoders are used to group similar patterns in medical images, which can lead to the discovery of new radiomic phenotypes. Finally, explainable AI (XAI) is emerging as a crucial trend in radiomics, with techniques like saliency maps or layer-wise relevance propagation (LRP) being used to make AI models more transparent, helping radiologists understand the rationale behind automated predictions.

In human disease detection, healthcare professionals collect a patient's medical history, including symptoms, family history, and lifestyle factors. Physical examinations are conducted to observe visible signs and symptoms, such as skin abnormalities, swelling, or abnormal sounds.
Laboratory Testing: Various laboratory tests are used to aid in disease diagnosis. These include blood tests, urine tests, tissue biopsies, imaging tests (X-rays, CT scans, MRIs), and molecular tests (PCR, gene sequencing). These tests help identify pathogens, measure specific biomarkers, or detect abnormal cell growth.
Medical Imaging: Imaging techniques like X-rays, CT scans, MRI, ultrasound, or PET scans are used to visualize internal structures, organs, and abnormalities within the body. These images help in detecting tumors, infections, fractures, or other structural changes associated with diseases.
Biomarker Analysis: Analysis of specific biomarkers, such as proteins, hormones, enzymes, or genetic markers, can aid in disease detection. Blood tests, for example, can measure levels of specific markers to identify diseases or monitor treatment responses.

Disease detection poses several challenges due to the complex nature of organisms' diseases and the diverse factors that can influence their detection. Some of the key challenges include:
Symptom Variability: Diseases can exhibit a wide range of symptoms, and these symptoms can vary depending on the plant species, the stage of disease progression, environmental conditions, and pathogen strains. This variability makes it challenging to develop universal detection methods that can accurately identify all types of diseases across different species.
Similar Symptoms: Certain diseases may exhibit symptoms that resemble those caused by other factors, such as nutrient deficiencies, environmental stress, or physical damage. Distinguishing between disease symptoms and non-disease-related symptoms can be difficult, requiring expert knowledge and careful analysis.
Pathogen Diversity: Diseases can be caused by a multitude of pathogens, including bacteria, fungi, viruses, and nematodes. Each pathogen has its own unique characteristics, life cycle, and infection patterns, making it challenging to develop generalized detection methods that can effectively detect all types of pathogens.
Early Detection: Early detection is crucial for effective disease management. However, in many cases, diseases remain undetected until visible symptoms appear, by which time significant damage may have already occurred. Developing techniques for early detection, such as molecular assays or remote sensing technologies, is a challenge that requires sensitive and specific detection methods.
Cost and Accessibility: Some disease detection methods, particularly those involving advanced laboratory techniques or specialized equipment, can be costly and require technical expertise. Making disease detection methods affordable, accessible, and user-friendly for farmers, medical researchers, and agronomists is an ongoing challenge.
Addressing these challenges requires interdisciplinary research, collaboration among scientists and industry experts, advancements in technology, and the integration of different detection approaches. Improving disease detection methods will help in early intervention, effective disease management, and sustainable practices.

Predicting the onset of diseases in plants/organisms using AI models presents several challenges that researchers and practitioners need to address. Some of the major challenges include:
1. Limited and unbalanced training data: AI models require large and diverse datasets for effective training. However, obtaining labeled data for plant diseases can be challenging due to limited availability and the need for expert annotation. Moreover, the distribution of data among different disease classes may be unbalanced, making it difficult for the model to generalize well across all diseases.
2. Variability in disease symptoms: Plant diseases can exhibit a wide range of symptoms, including variations in color, texture, shape, and size. Capturing and representing this variability in training data can be complex. AI models must be trained to recognize and differentiate between subtle disease symptoms and other factors that might affect plant appearance, such as nutrient deficiencies, environmental stress, or physical damage.
3. Transferability to new environments: Models trained on data from specific geographic regions or plant species may struggle to generalize well when deployed in new environments or on different plant varieties. The variability in environmental conditions, plant genetics, and disease prevalence requires robust models that can adapt and generalize across diverse settings.
4. Early detection and false positives: Detecting diseases in their early stages is crucial for effective disease management. However, early symptoms may be subtle and easily missed, leading to delayed or inaccurate predictions. AI models need to be sensitive enough to identify these early indicators while minimizing false positives, which can result in unnecessary interventions or increased costs.
5. Scalability and real-time processing: Deploying AI models for large-scale agricultural applications requires efficient processing and scalability. Real-time or near real-time prediction is crucial to enable timely interventions. Balancing the computational demands of AI algorithms with the need for rapid analysis poses a challenge, especially when dealing with high-resolution images or large datasets.
6. Interpretability and trust: AI models are often considered black boxes, making it challenging to understand the reasoning behind their predictions. In the agricultural domain, it is crucial to have transparent and interpretable models to gain trust and facilitate decision-making. Ensuring explainability and interpretability of AI models is important to gain acceptance and adoption in plant disease prediction.

From an image analysis perspective, CIELAB, also known as LAB color space or Lab* color space, is a color model used to represent colors in a device-independent and perceptually uniform manner. It was developed by the International Commission on Illumination (CIE) as a standard color space for accurately describing and comparing colors.

### The CIELAB Color Model Consists of Three Components:

L* (Lightness): The L* component represents the perceived lightness or brightness of a color. It ranges from 0 to 100, where 0 represents black and 100 represents white. The midpoint of the scale, L*=50, is considered a neutral gray.
a* (Green-Red axis): The a* component represents the position along the green-red axis. Positive values indicate a shift towards red, while negative values indicate a shift towards green. The range of a* typically extends from −128 to +127.
b* (Blue-Yellow axis): The b* component represents the position along the blue-yellow axis. Positive values indicate a shift towards yellow, while negative values indicate a shift towards blue. The range of b* typically extends from −128 to +127.
The CIELAB color space is designed to be perceptually uniform, meaning that an equal numerical difference in the Lab* values corresponds to a similar perceptual difference in color across the entire color space. This makes it useful for various color-related applications, such as color management, color matching, and color difference calculations.

Clustering can be used to analyze CIELAB data by grouping similar colors together based on their perceptual similarities in the LAB color space. Clustering is a technique used in unsupervised machine learning to group similar data points together based on their inherent similarities or patterns. The objective is to discover natural groupings or clusters within the data, where data points within the same cluster are more similar to each other compared to those in other clusters. Clustering is widely used in various domains, including data analysis, pattern recognition, image processing, and customer segmentation.
Different types of clustering algorithms exist, and they can be categorized based on various factors, such as the underlying algorithmic approach, the shape of the clusters, or the assumptions made during the clustering process. Here are some commonly used types of clustering:
K-Means Clustering: It is a centroid-based clustering algorithm. It aims to partition the data into K clusters, where K is a predetermined number. The algorithm iteratively assigns data points to the nearest cluster centroid and updates the centroids until convergence. K-Means assumes that clusters are spherical and have similar sizes.
Hierarchical Clustering: This type of clustering creates a hierarchy of clusters using a bottom-up (agglomerative) or top-down (divisive) approach. In agglomerative hierarchical clustering, each data point initially represents a single cluster, and then clusters are successively merged based on their similarity until a single cluster containing all the data points is formed. Divisive hierarchical clustering works in the opposite way, starting with a single cluster and iteratively splitting it into smaller clusters. The result is a tree-like structure called a dendrogram that shows the relationship between the clusters at different levels.
Density-Based Clustering: Density-based clustering algorithms, such as DBSCAN (Density-Based Spatial Clustering of Applications with Noise), group together data points that are densely connected to each other. It identifies clusters as regions of high-density separated by regions of low-density. DBSCAN does not require a predetermined number of clusters and can discover clusters of arbitrary shapes.
Gaussian Mixture Models (GMM): GMM is a probabilistic model that assumes the data points are generated from a mixture of Gaussian distributions. The algorithm aims to estimate the parameters of these distributions and assign data points to the most likely cluster based on their probabilities. GMM can handle data with complex distributions and can identify clusters with different shapes and sizes.
Fuzzy Clustering: Fuzzy clustering allows for the partial membership of data points to multiple clusters. Instead of hard assignments, fuzzy clustering assigns membership values to data points, indicating the degree of association with each cluster. Fuzzy C-Means (FCM) is a well-known fuzzy clustering algorithm.
Model-Based Clustering: Model-based clustering algorithms, such as Expectation-Maximization (EM), assume that the data is generated from a specific statistical model. These algorithms estimate the parameters of the model and assign data points to clusters based on their likelihood under the model. The number of clusters may be automatically determined using techniques like the Bayesian Information Criterion (BIC).

CIELAB color space enhances tissue detection in medical imaging by offering improved visual contrast and color differentiation compared to traditional grayscale images. This color space separates lightness (L*) from color components (a* and b*), which correspond to red-green and blue-yellow color channels, respectively. This separation allows for more precise visualization of subtle variations in tissue structures, which might be missed in grayscale. By focusing on both lightness and color information, CIELAB enables medical professionals to detect early signs of diseases, such as small lesions or abnormalities that might otherwise blend into surrounding tissue.

Additionally, CIELAB's perceptually uniform nature means that small changes in image data correspond to perceptible changes in color, enhancing the detection of minute tissue differences. This makes CIELAB particularly useful for identifying anomalies like tumors or tissue degeneration, where variations in texture or intensity are key diagnostic indicators. By applying pseudo-coloring techniques with CIELAB, these differences are made more apparent, aiding both machine learning models and clinicians in detecting abnormalities. When combined with CNNs and machine learning algorithms, CIELAB-transformed images provide more accurate input data, improving the performance of disease prediction models by highlighting subtle tissue variations

### SUMMARY OF THE INVENTION

The present invention provides a two-step method for predicting the onset of human diseases by analyzing medical images using a combination of Convolutional Neural Networks (CNN) and pseudo-color transformation in the CILELAB color space. The method first utilizes a CNN for an initial down-selection based on key radiomic features from grayscale medical images, followed by a detailed analysis using pseudo-color images in the CILELAB color space to refine disease detection. This two-step process allows for early and more accurate disease detection.

The method comprises the following steps:

### Step 1: Initial Down-Selection Using CNN

Data Acquisition: Capturing medical images and storing them in digital grayscale format.
Radiomic Feature Extraction: Applying a CNN-based algorithm to extract key disease-relevant features from the grayscale images. This initial CNN analysis performs a down-selection to identify areas of interest that may indicate early signs of disease.

### Step 2: Disease Detection Using CILELAB Color Space

Image Preprocessing: Converting the grayscale medical images that passed the CNN down-selection into a pseudo-colored representation using the CILELAB color space.
Detailed Analysis: The pseudo-colored images are analyzed to enhance subtle variations in the tissue structure, and a machine learning-based classifier predicts the likelihood of disease onset. This step provides a refined prediction of disease presence based on enhanced visual features.
Output: Generating a prediction report that highlights areas of interest in the medical image and provides a likelihood score for disease presence.

### DETAILED DESCRIPTION OF THE INVENTION

The CIELAB color space, also known as Lab*, was defined by the International Commission on Illumination in 1976 and expresses color using three values: L* for perceptual lightness, and a* and b* for the four unique colors of human vision-red, green, blue, and yellow. It was designed as a perceptually uniform space, meaning that a given numerical change corresponds to a similar perceived change in color. An important aspect of CIELAB is its device-independence, meaning that the colors it defines are not tied to specific devices like monitors or printers but are based on the CIE standard observer, an average of results from color matching experiments under controlled laboratory conditions. The CIELAB space is three-dimensional and encompasses the full range of human color perception, or gamut, based on the opponent color model. In this model, red and green form an opponent pair, as do blue and yellow. The lightness value, L*, also known as “Lstar,” ranges from 0 (black) to 100 (white). The a* axis ranges from negative (green) to positive (red), while the b* axis ranges from negative (blue) to positive (yellow). Although the a* and b* axes are theoretically unbounded, practical implementations often restrict these values, typically between −128 and 127, depending on the reference white used.

The hue of a color in this space can be quantified by its hue angle, which is calculated using the formula:

$${h}_{ab} = arctan ( \frac{{b}^{*}}{{a}^{*}} ) . \qquad Equation 1$$

Chroma, representing color saturation, is calculated as:

$${C}_{ab} = \sqrt{{a}^{* 2} + {b}^{* 2} .} \qquad Equation 2$$

Colors with high chroma are perceived as clear and bright, while duller colors have low chroma. The CIELAB color space plays a key role in disease detection in this method.

The process begins with Step 1: Initial Down-Selection Using CNN, where medical images such as CT, MRI, or X-rays are captured and stored in grayscale format. A Convolutional Neural Network (CNN), trained on large datasets, is used to extract relevant disease-related features by analyzing image attributes like texture, shape, and intensity variations. This first step narrows down regions of interest for further analysis.

In Step 2: Disease Detection Using CIELAB Color Space, the grayscale regions identified in Step 1 are converted into the CIELAB color space to enhance visibility of subtle tissue differences. The separation of lightness (L*) from color components (a* and b*) allows better differentiation of early disease markers. A machine learning classifier is then applied to the pseudo-colored images to predict the likelihood of disease onset, resulting in a more detailed and accurate analysis compared to grayscale images alone.

Finally, the system generates a comprehensive output report that includes:
A heatmap highlighting the areas of interest identified by both the CNN and CIELAB analysis.
A probability score predicting the likelihood of disease presence.
Recommendations for further clinical review or diagnostic steps based on the results.

This two-step method combining CNN-based feature extraction with CIELAB color space analysis allows for early and accurate disease detection, enhancing the predictive power of medical imaging.

### The Advantages of the Invention are

Enhanced Early Detection: The two-step approach facilitates early disease identification by leveraging CNN technology to pinpoint potential areas of interest, followed by a detailed pseudo-color analysis that offers more precise and refined predictions.
Superior Visualization: The transformation of grayscale images into the CIELAB color space significantly improves the detection of subtle tissue abnormalities, making early disease markers more discernible.
Fully Automated Workflow: This method streamlines the entire diagnostic process, automating feature extraction through CNN and refining disease detection using pseudo-color imaging, thereby minimizing the need for manual interpretation.
Tailored Diagnostics: The approach can be customized to detect a wide range of diseases, offering a versatile and adaptable solution across various medical conditions.

### BRIEF DESCRIPTION OF THE DRAWINGS

Reference is made to the accompanying drawings in which is shown an illustrative embodiment of the invention, from which its novel features and advantages will be apparent.

FIG. 1 represents a comprehensive workflow from MRI scanning to disease diagnosis and presentation

FIG. 2 represents a Convolutional Neural Network (CNN) architecture designed to process medical imaging data such as CT scans, MRI scans, or X-rays to detect and predict the presence of anomalies, such as tumors

FIGS. 3, 4, 5, 6 and 7 are scans depicting different stages of Glioma.

Referring to FIGS. 8, 9, 10, 11 and 12 are MRI scans depicting different stages of meningioma.

Referring to FIG. 13a, is a MRI scan for Glioma tumor, and FIG. 13b is its pseudo-color chart using CIELAB.

Referring to FIG. 14a, is a MRI scan for healthy condition, and FIG. 14b is its pseudo-color chart using CIELAB.

Referring to FIG. 15a, is a MRI scan for Meningioma tumor, and FIG. 15b is its pseudo-color chart using CIELAB.

Referring to FIG. 16a, is a MRI scan for Pituitary tumor, and FIG. 16b is its pseudo-color chart using CIELAB.

FIG. 17 represents the CIELAB color space in a circular diagram, showcasing how different colors are mapped based on their hue and chroma values.

FIG. 18 is a visual of a user interface screen for a medical diagnosis system showing the result of a brain tumor diagnosis. It highlights the MRI scan with a detected tumor, probability percentages, and options for saving the report or moving to the next scan

### DESCRIPTION OF THE PREFERRED EMBODIMENTS

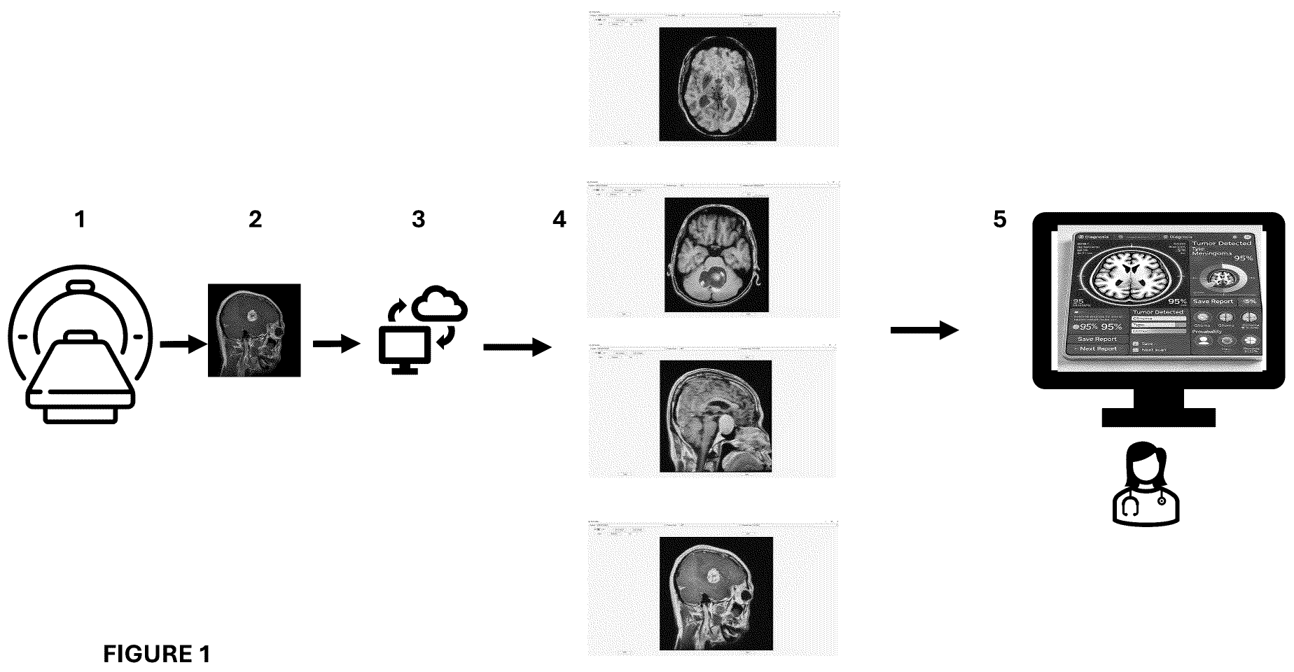

In FIG. 1 a comprehensive workflow from MRI scanning (Step 1) to tumor diagnosis and presentation (Step 5). The process involves scanning the brain using MRI (Step 1), analyzing the images using a CNN model (Step 3), enhancing the visualization with CIELAB and pseudo-coloring (Step 4), and delivering the results to a medical professional (Step 5). The combination of machine learning, color space transformation, and automated prediction allows for accurate, efficient detection and classification of brain tumors. This system enhances diagnostic accuracy by leveraging advanced image processing techniques.

The steps, as labeled in the figure, describe the process as follows:

### Step 1: MRI Scan (Labeled “1”)

The process begins with the MRI machine, which scans the patient's brain to capture detailed images. These MRI scans provide crucial structural information used to detect abnormalities like tumors.

### Step 2: MRI Image (Labeled “2”)

Once the MRI scan is complete, the output is a brain MRI image, showing various cross-sections of the patient's brain. These images serve as the input for further analysis in the diagnostic system. The scan shows the brain's internal structures, which will later be used by the CNN for feature extraction and classification.

### Step 3: CNN Model (Labeled “3”)

The MRI scan image is then fed into a Convolutional Neural Network (CNN) model. The CNN processes the scan through several layers, extracting key features such as edges, shapes, and textures. The CNN identifies patterns that suggest the presence of anomalies (e.g., a tumor). This stage performs automated analysis to predict whether a tumor is present and estimates the probability of its existence.

### Step 4: CIELAB Color Space and Pseudo-Coloring (Labeled “4”)

The processed output from the CNN model is further analyzed using the CIELAB color space combined with pseudo-coloring techniques.
CIELAB: Breaks down the image into L (Lightness), A (Green-Red), and B (Blue-Yellow) channels to enhance subtle differences between healthy and tumor-affected tissues.
Pseudo-Coloring: Different color gradients are applied to grayscale images to make the tumor region more visible. These enhanced images highlight the tumor location, shape, and size in distinct color tones for easier identification and diagnosis.

### Step 5: Results Seen by the Doctor (Labeled “5”)

Finally, the results are displayed on a screen for the doctor or medical professional to review. The screen shows the processed MRI images, tumor detection information, the type of tumor (e.g., glioma, meningioma), the predicted probability (e.g., 95%), and suggested next steps (e.g., saving the report). This interface allows the doctor to make informed decisions about diagnosis and treatment based on the advanced image processing and analysis performed by the system.

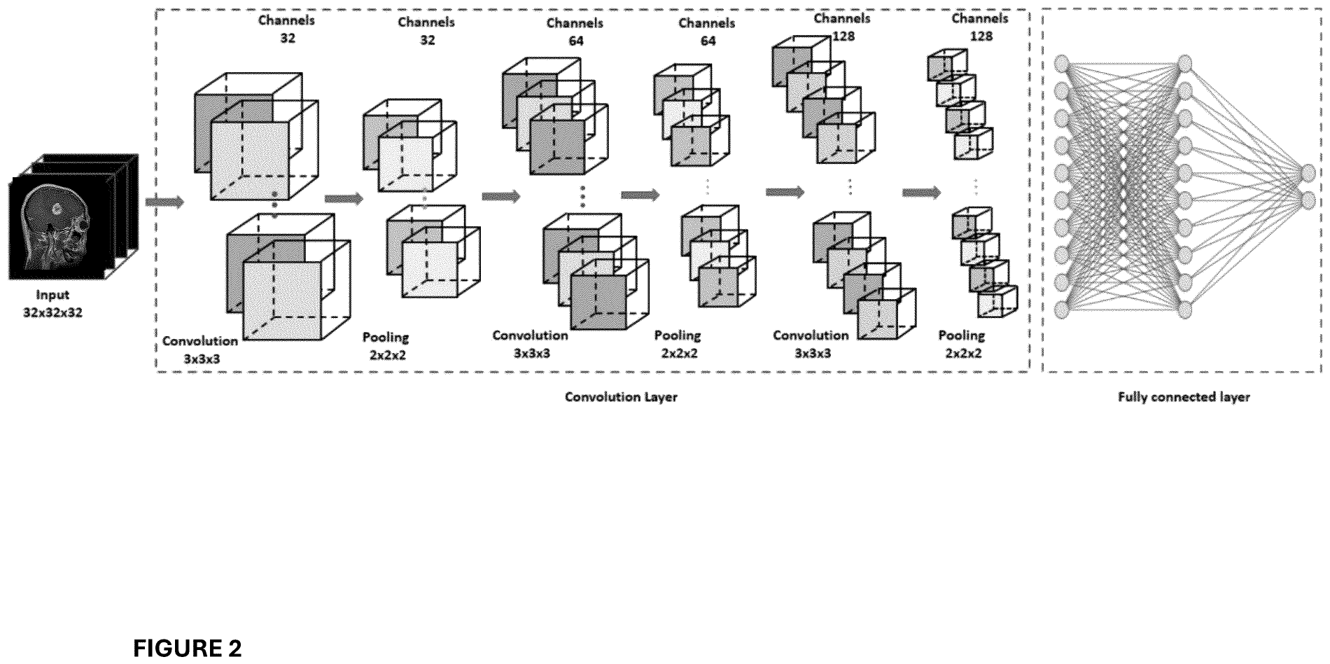

This FIG. 2 represents a Convolutional Neural Network (CNN) architecture designed to process medical imaging data such as CT scans, MRI scans, or X-rays to detect and predict the presence of anomalies, such as tumors. The CNN is composed of several layers, each playing a distinct role in feature extraction, data dimensionality reduction, and final prediction.

### Input Layer:

Input Image (32×32×32): The process begins with an input image (for example, a slice of a 3D brain MRI or CT scan) of dimensions 32×32×32. This input image represents the scanned data that the CNN will analyze to detect patterns or anomalies. The image is divided into multiple channels, allowing the CNN to focus on different aspects of the scan for a detailed analysis.

### Convolutional Layers:

Convolution 3×3×3 (Channels=32): The input image is passed through the first convolutional layer. This layer applies 3×3×3 filters across the entire image, detecting local patterns such as edges, textures, and other important features in small sections of the image. These features are passed to the next layer, generating 32 channels or feature maps, each highlighting different aspects of the scan.
Pooling 2×2×2 (Channels=32): Following the convolution, a pooling layer (specifically max pooling) reduces the dimensions of the feature maps. Pooling ensures that only the most important features (such as the most prominent edges) are retained, while reducing the overall size of the data, making the network computationally more efficient without losing essential information.
Convolution 3×3×3 (Channels=64): Another convolutional layer is applied, this time increasing the number of channels to 64. This layer builds on the previously detected features by focusing on more complex patterns or shapes that may indicate the presence of a tumor or other anomaly.
Pooling 2×2×2 (Channels=64): Again, pooling reduces the data size while maintaining the key features, preparing the feature maps for further analysis.
Convolution 3×3×3 (Channels=128): A third convolutional layer with more filters (128 channels) refines the feature detection. This layer looks for even higher-level structures or patterns that might represent specific types of anomalies, such as irregular tissue growth or abnormal shapes typical in tumors.
Pooling 2×2×2 (Channels=128): The final pooling layer again reduces the size of the feature maps to retain the most important features extracted from the original scan.

### Fully Connected Layer:

After the convolution and pooling layers, the extracted features are flattened and passed into the fully connected layer. This layer is where the learned features are synthesized to make predictions. Each node in the fully connected layer is connected to every node in the previous layer, allowing the network to combine and evaluate all the extracted features.

### Prediction Output:

The fully connected layer generates a prediction, which could represent the probability of the presence of a specific anomaly (e.g., tumor presence). For example, the system may predict that there is a 95% probability of a tumor based on the features extracted from the scan.

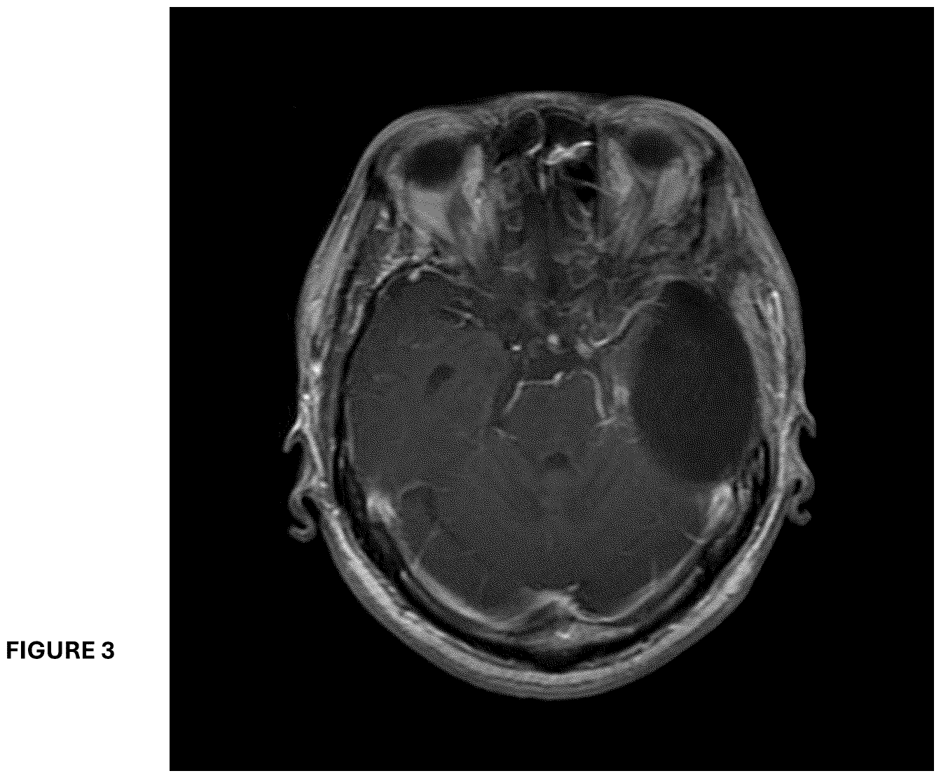

Referring to FIGS. 3, 4, 5, 6 and 7 are MRI scans depicting different stages of Glioma.

Referring to FIGS. 8, 9, 10, 11 and 12 are MRI scans depicting different stages of meningioma.

Referring to FIG. 13a, is a MRI scan for Glioma tumor, and FIG. 13b is its pseudo-color chart using CIELAB.

Referring to FIG. 14a, is a MRI scan for healthy condition, and FIG. 14b is its pseudo-color chart using CIELAB.

Referring to FIG. 15a, is a MRI scan for Meningioma tumor, and FIG. 15b is its pseudo-color chart using CIELAB.

Referring to FIG. 16a, is a MRI scan for Pituitary tumor, and FIG. 16b is its pseudo-color chart using CIELAB.

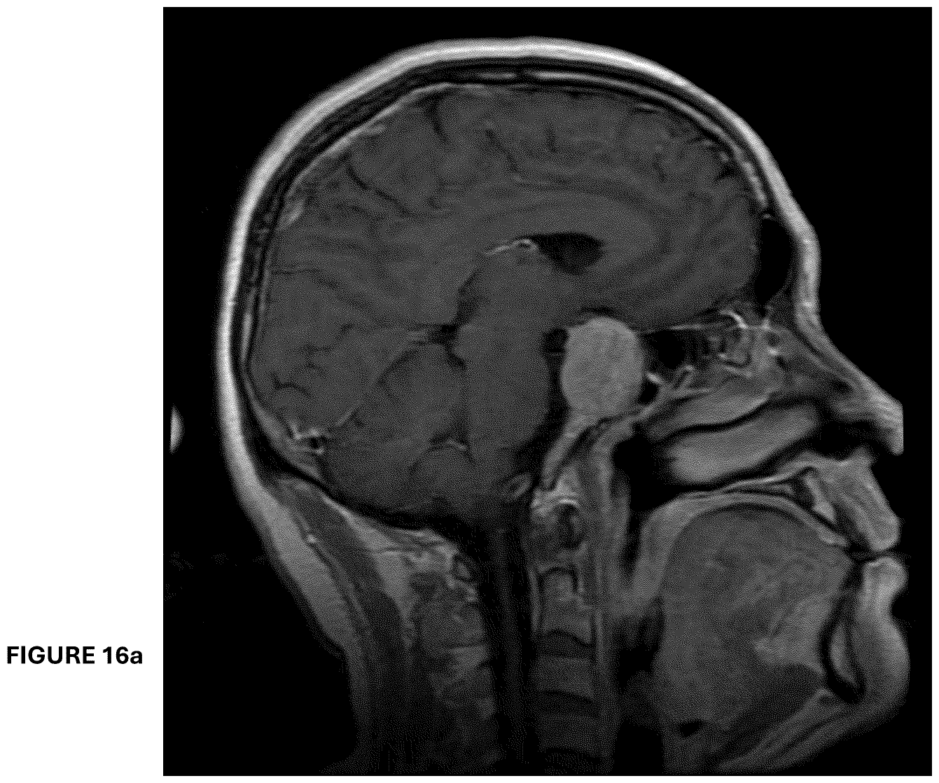

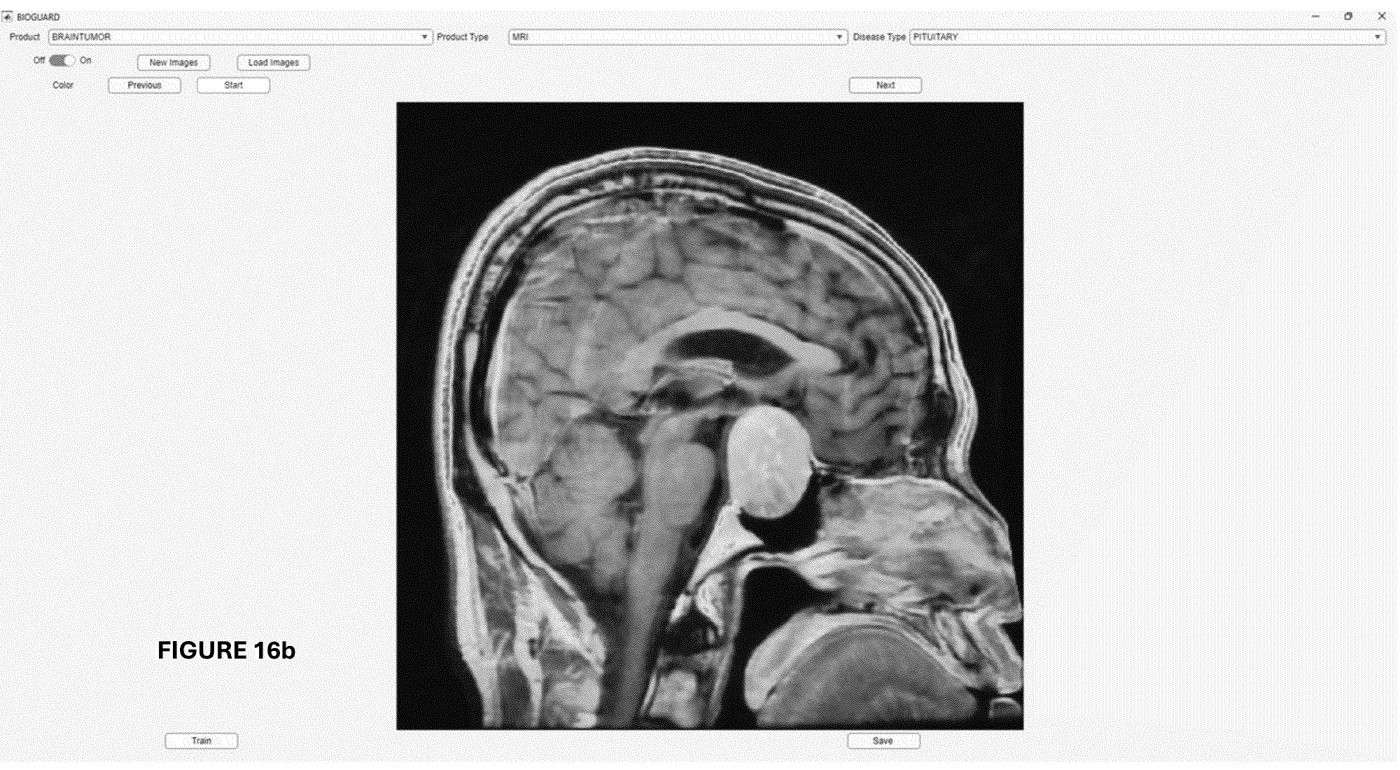

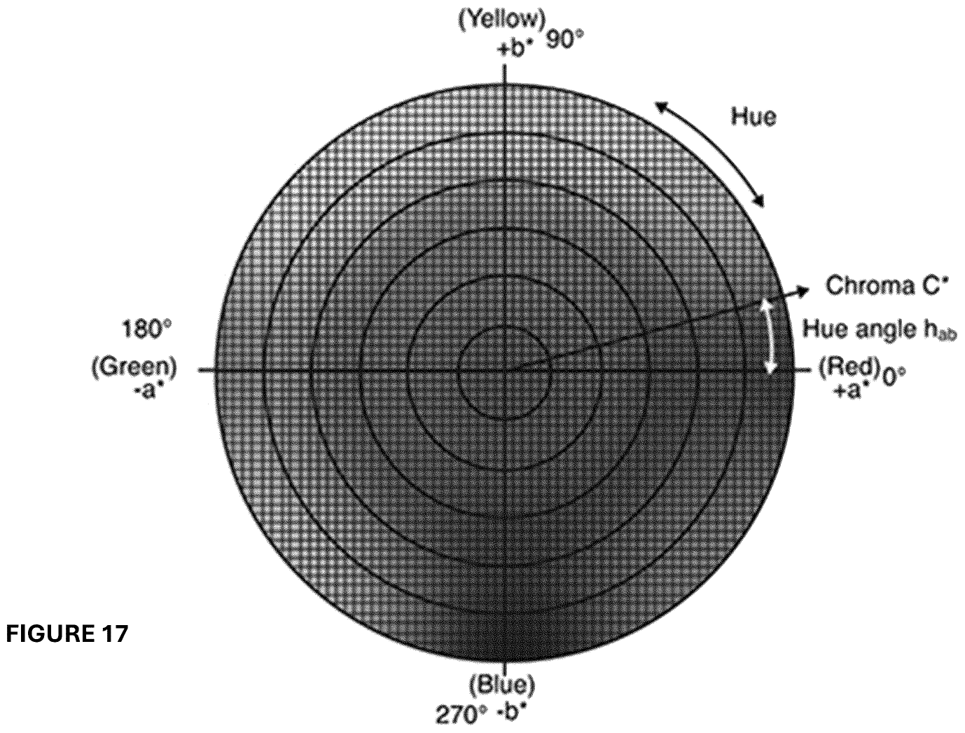

FIG. 17 represents the CIELAB color space in a circular diagram, showcasing how different colors are mapped based on their hue and chroma values.

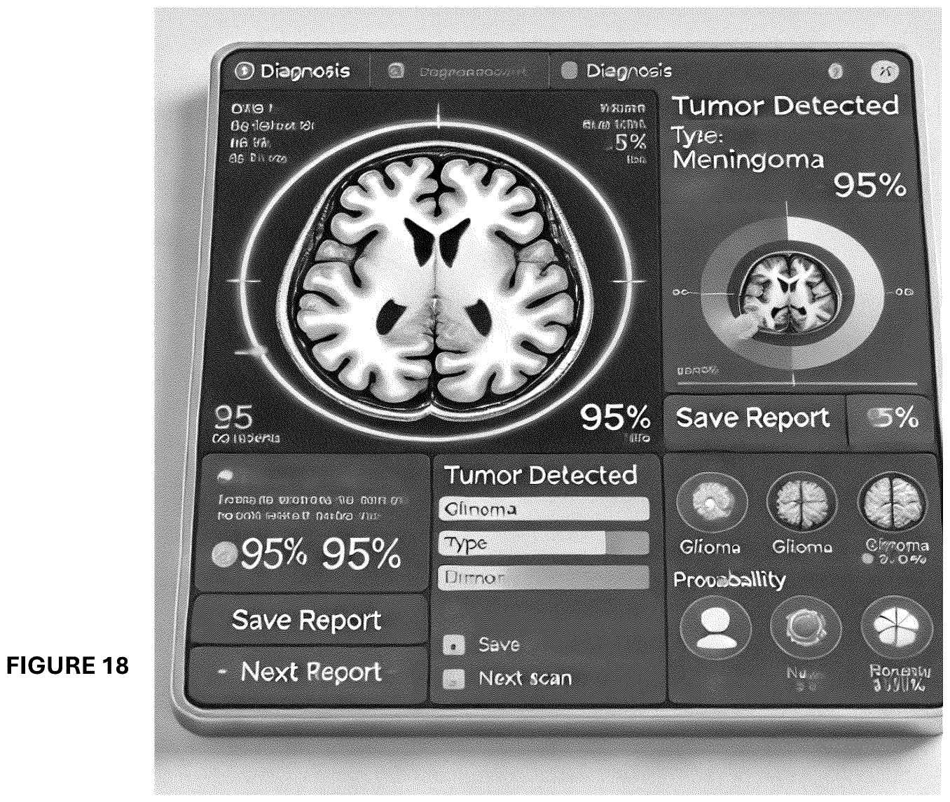

FIG. 18 is a visual of a user interface screen for a medical diagnosis system showing the result of a brain tumor diagnosis. It highlights the MRI scan with a detected tumor, probability percentages, and options for saving the report or moving to the next scan.

The invention will now be illustrated, but not limited, by reference to the specific embodiments described in the following example.

### Example

In this Example, Convolutional Neural Networks (CNNs) are first leveraged to predict the presence and probability of a brain tumor, followed by validation using CIELAB color space and pseudo-coloring techniques to classify the specific type of tumor, such as pituitary adenomas, meningiomas, or gliomas.

The first step involves utilizing a pre-trained CNN model to analyze MRI brain scans for indications of tumor presence. CNNs, as powerful deep learning models, can identify intricate patterns in medical images that may indicate abnormal growths or structural anomalies. An MRI scan is fed into the CNN, which processes it through several layers: Convolutional Layers scan the image, applying filters to extract features like edges, contours, and textures. Pooling Layers reduce the size of the data while retaining essential features, making the network more efficient. Fully Connected Layers analyze the extracted features and generate a prediction, which includes the probability that a tumor is present. For example, in this step, the CNN may predict a 95% likelihood of the presence of a tumor without yet identifying its specific type. At this stage, the CNN confirms the presence of abnormal structures but does not differentiate between types, such as pituitary adenomas, meningiomas, or gliomas.

Once the CNN has identified a high probability of a tumor, the results are documented in a simple to use deep learning library. The next step is validating and classifying the type of tumor using CIELAB color space and pseudo-coloring techniques. This two-step process helps distinguish between different types of tumors by enhancing the visualization of tissue characteristics that might otherwise be subtle or difficult to detect. The first step is CIELAB Color Space Conversion, where the MRI scan is transformed into the CIELAB color space. This model provides a more perceptually uniform representation than traditional grayscale or RGB formats. The image is broken down into three components: L (Lightness), which emphasizes the contrast between dark and light regions, making tumors more visually prominent, and A (Green-Red) and B (Blue-Yellow) channels, which further differentiate tumor and healthy tissue by bringing out variations in texture and density. This conversion highlights key visual features that are essential for identifying the specific type of tumor, allowing for more precise classification.

Next, Pseudo-Coloring for Feature Enhancement is applied to further refine the classification. In this step, grayscale intensities from the MRI are mapped to different colors, making it easier to differentiate the tumor from surrounding tissue. For instance, a pituitary tumor may appear in one color gradient, while a glioma may show up in another, helping the system classify the tumor type based on color patterns. The pseudo-color representation, combined with the enhanced detail provided by CIELAB, enables the system to accurately predict the specific tumor type. This combination of CNN for initial detection and CIELAB with pseudo-coloring for classification ensures high accuracy in both tumor detection and type identification. Refer to FIGS. 13b, 14b, 15b and 16b.

Table 1 presents the tumor predictions based on the described method for detecting gliomas.

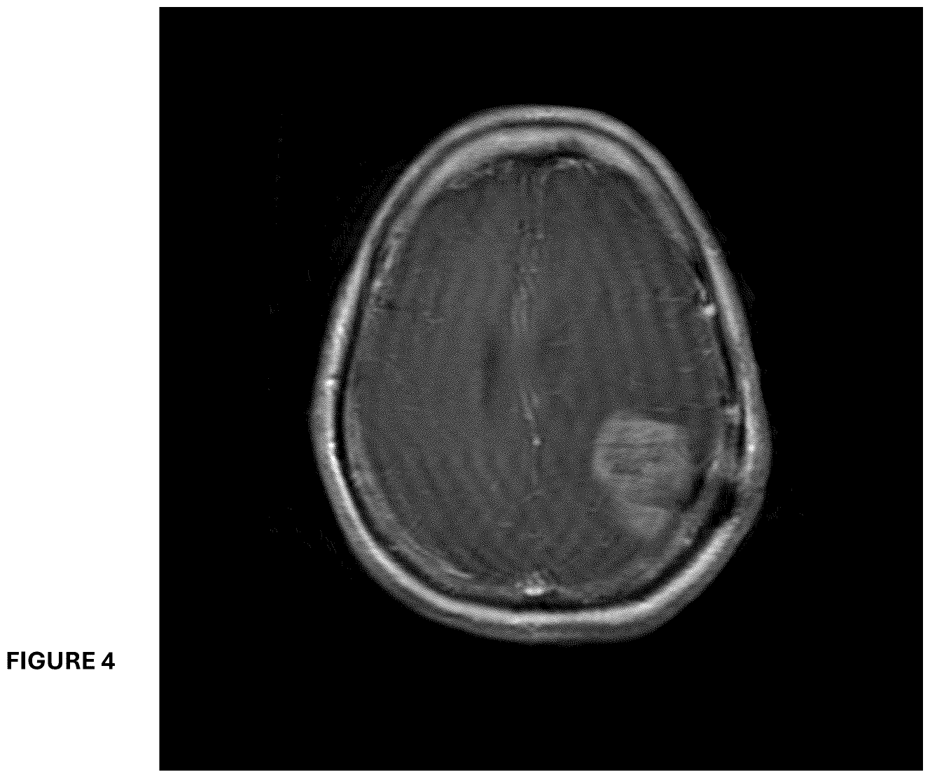

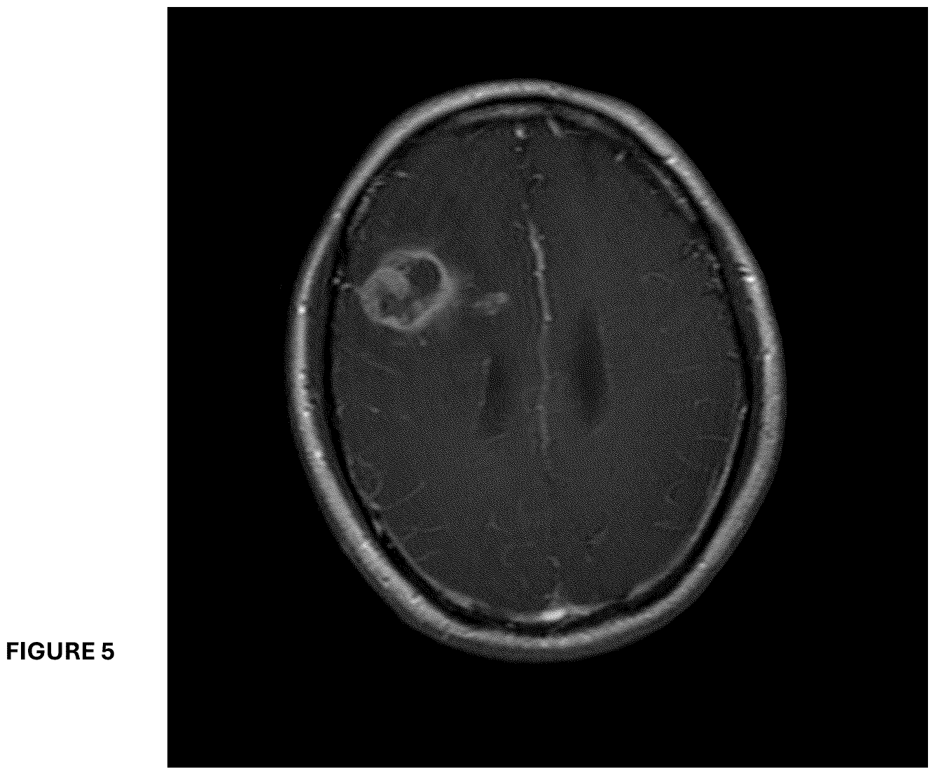

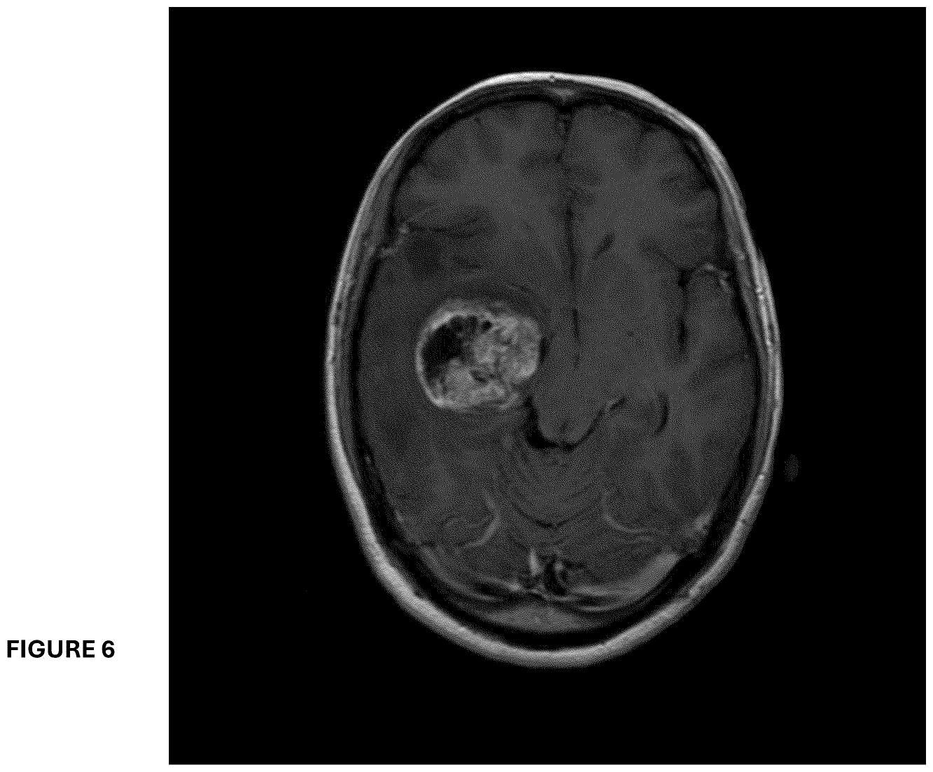

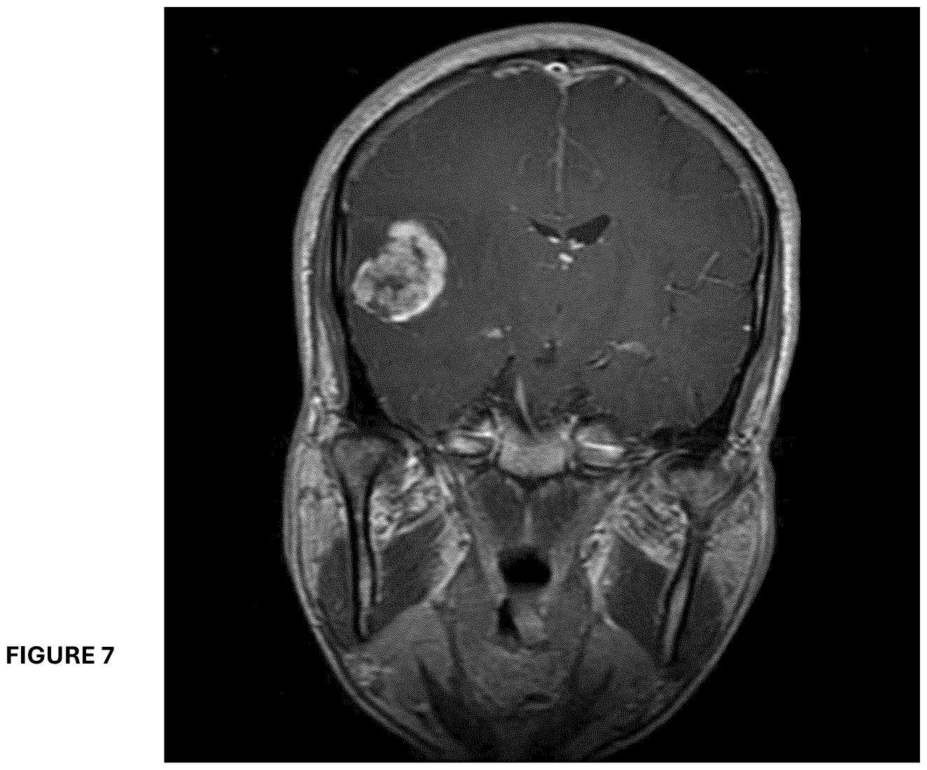

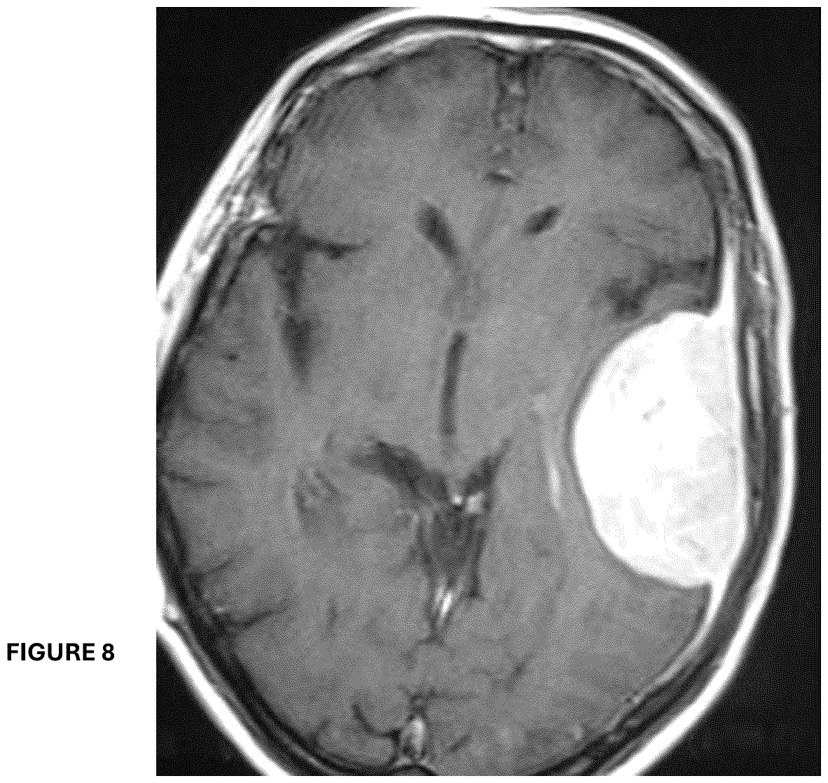

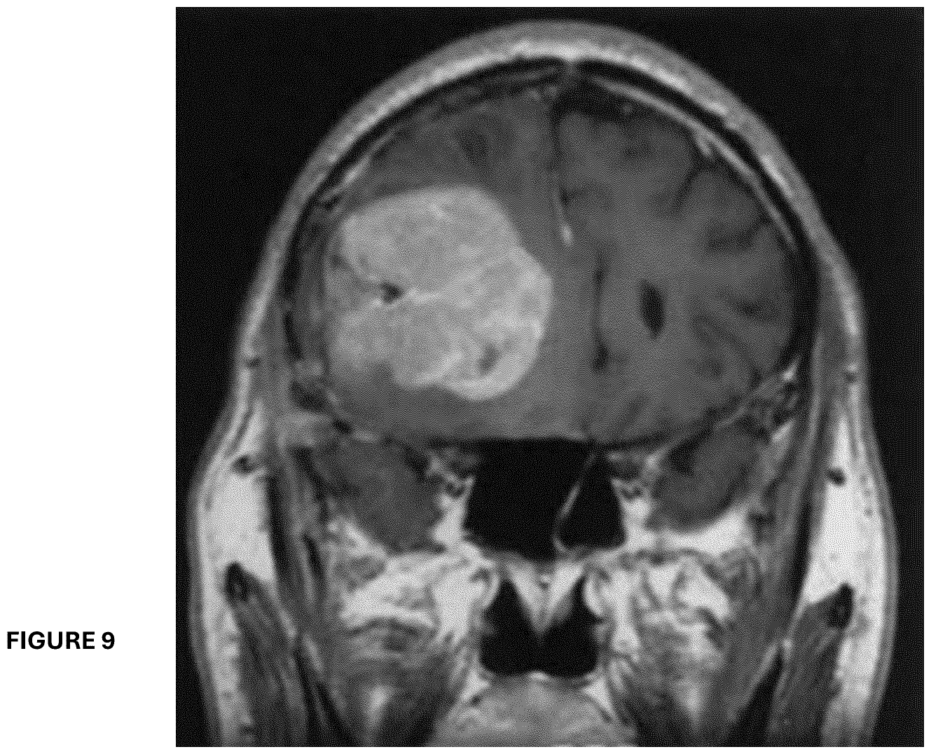

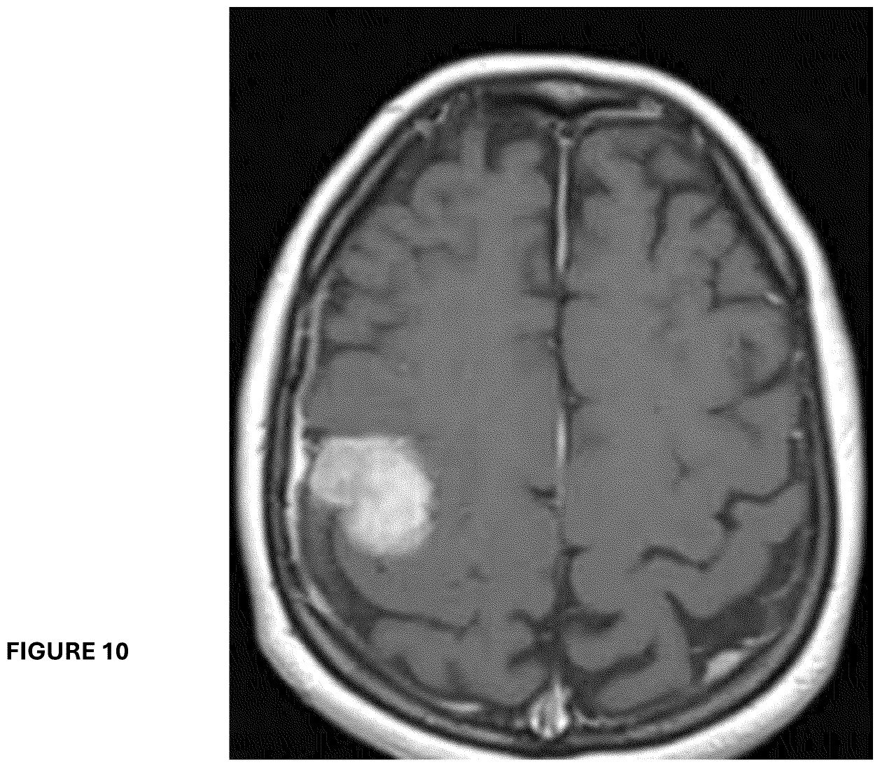

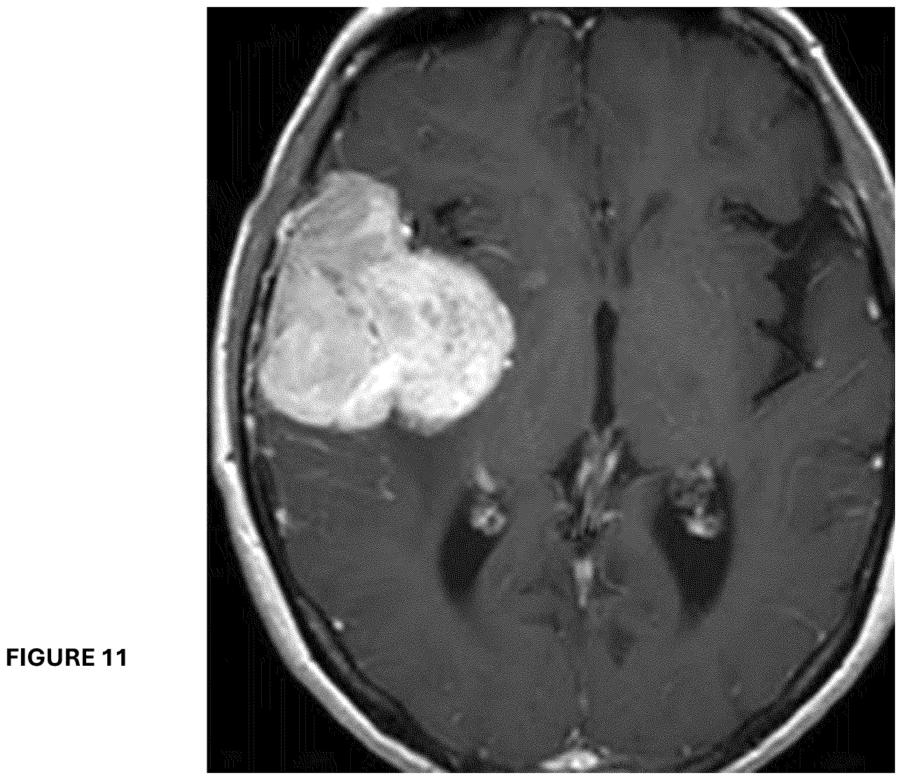

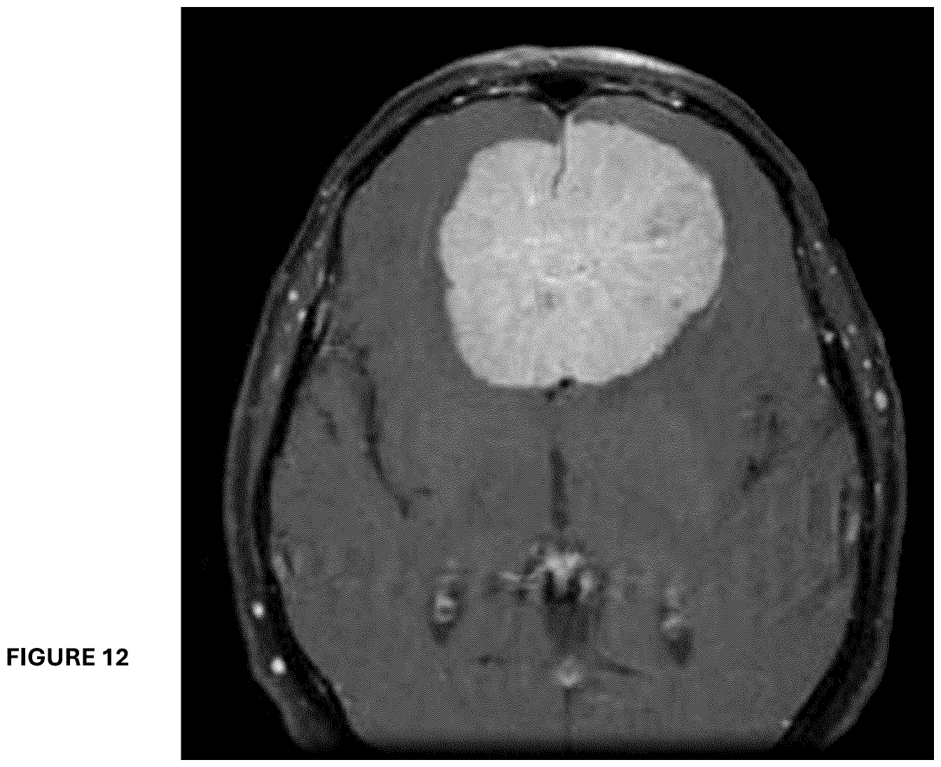

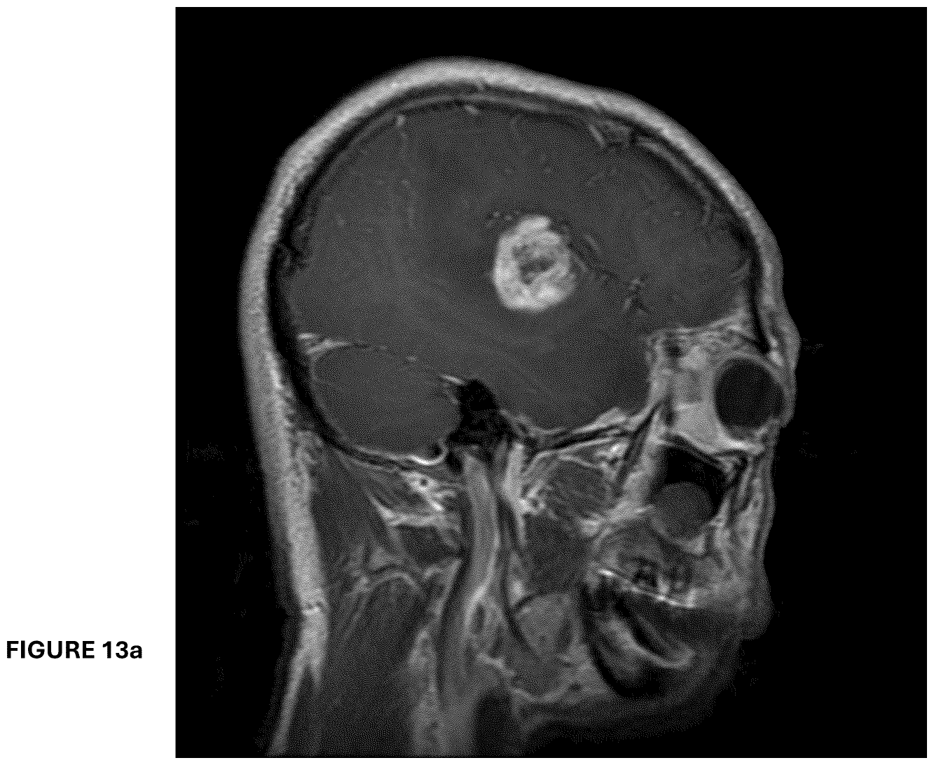

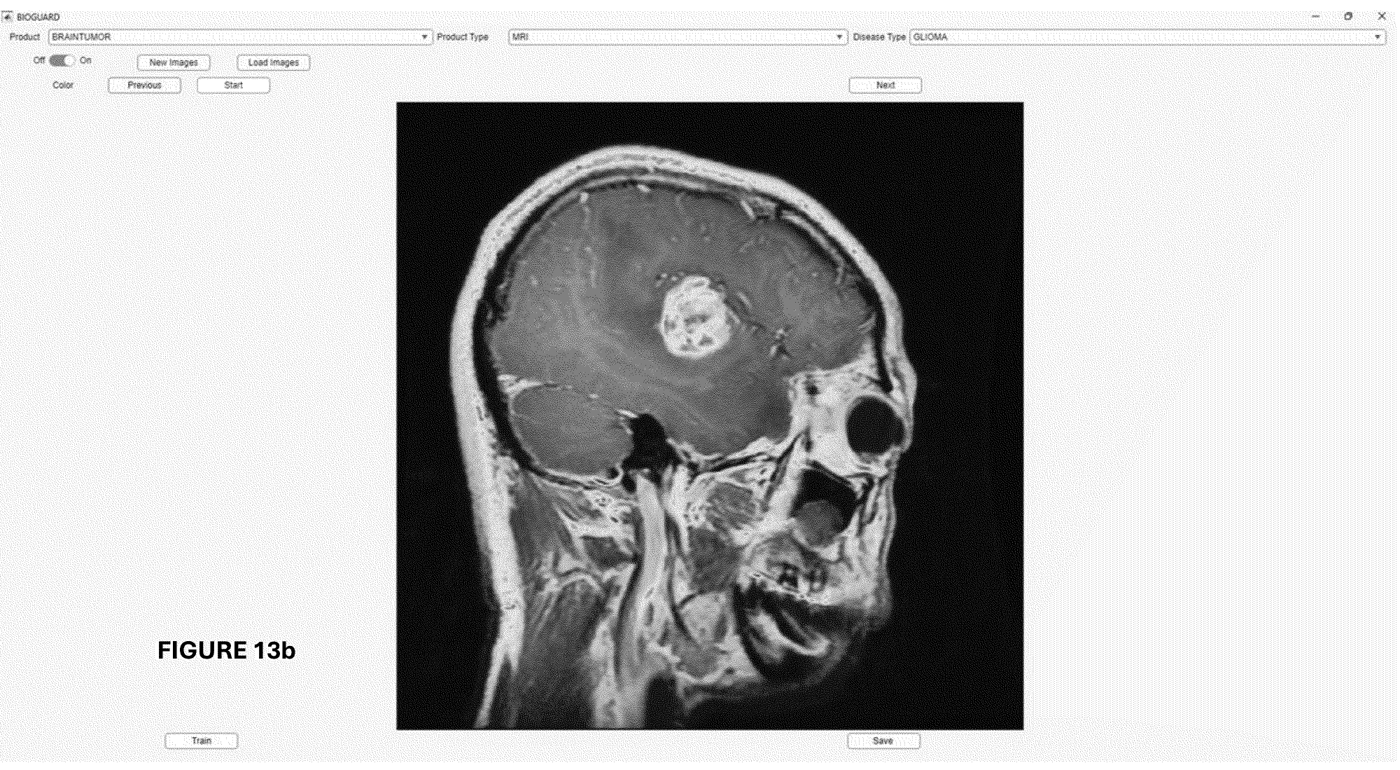

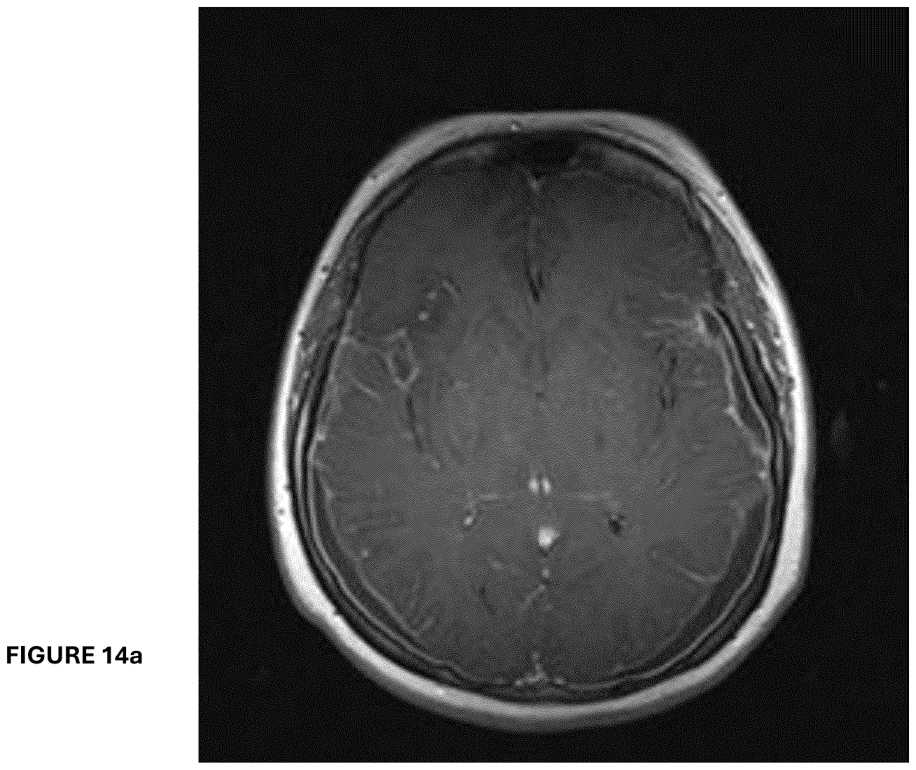

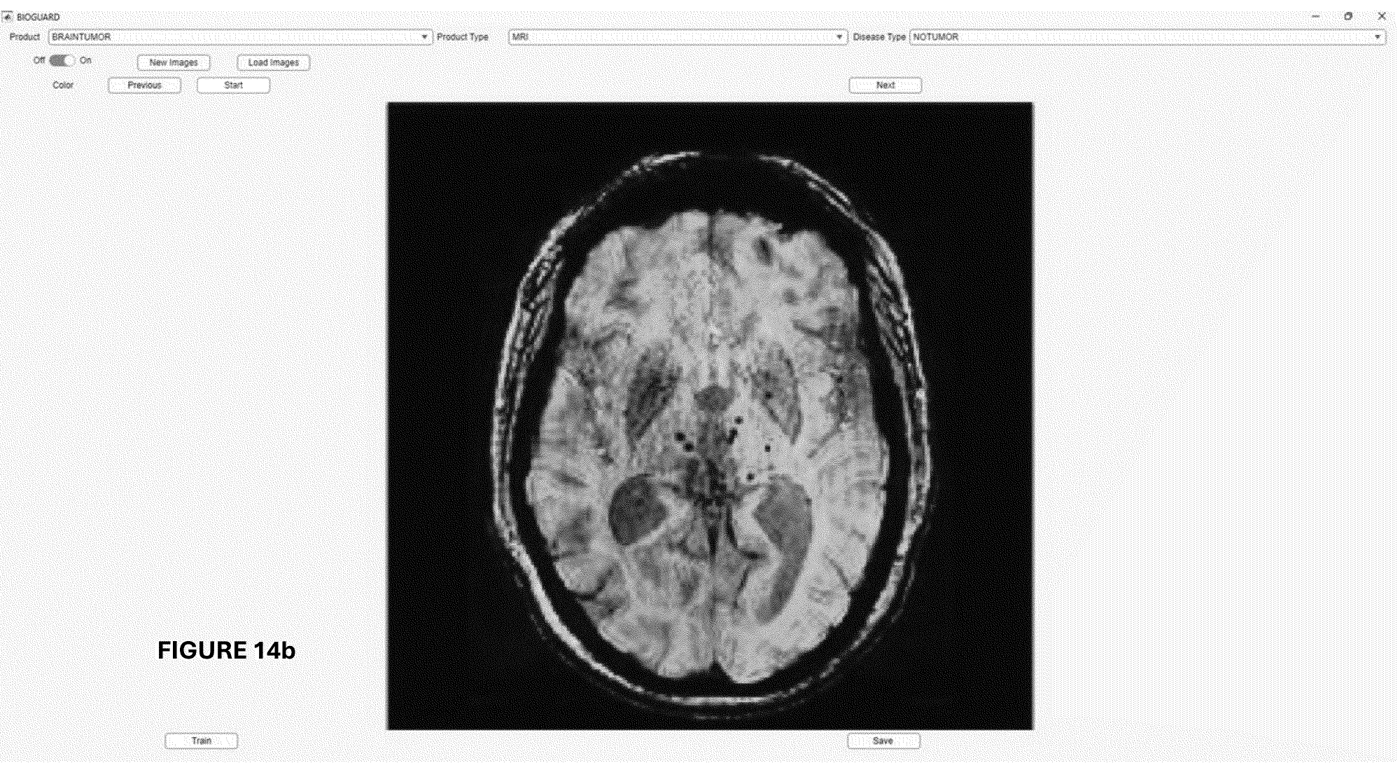

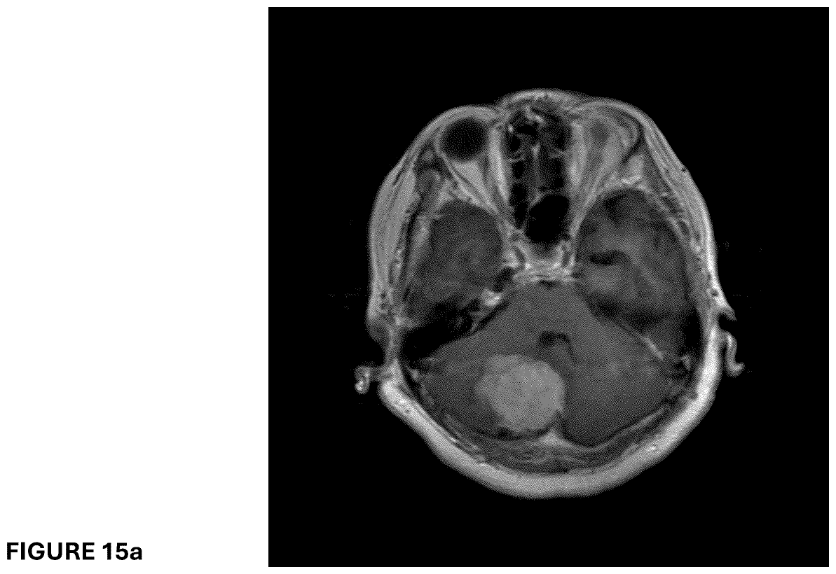

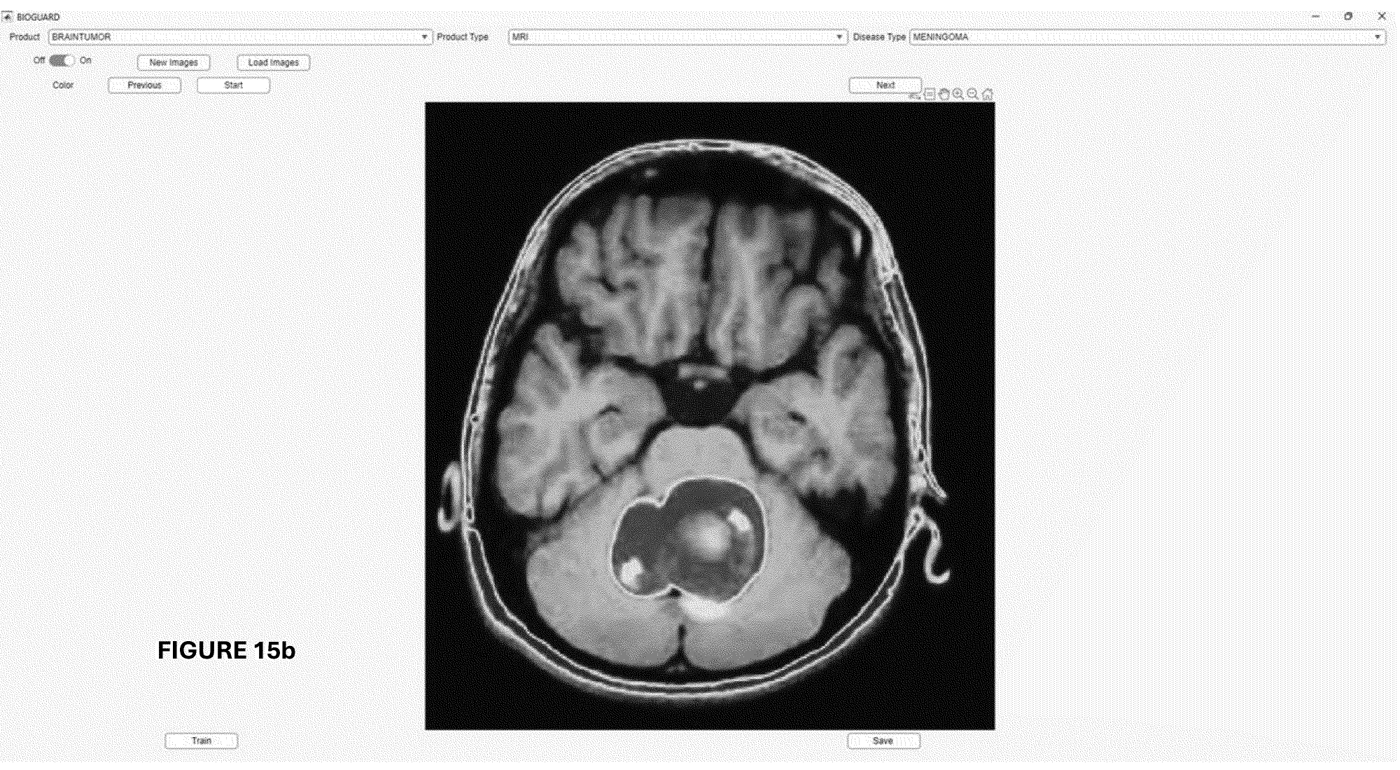

TABLE 1
PREDICTIONS FOR GLIOMA

| Label | Probability | Corresponding Image |
|---|---|---|
| glioma | 83.517 | FIG. 3 |
| pituitary | 16.305 | FIG. 3 |
| meningioma | 0.137 | FIG. 3 |
| notumor | 0.041 | FIG. 3 |
| glioma | 98.038 | FIG. 4 |
| pituitary | 1.505 | FIG. 4 |
| meningioma | 0.394 | FIG. 4 |
| notumor | 0.063 | FIG. 4 |
| glioma | 99.883 | FIG. 5 |
| pituitary | 0.101 | FIG. 5 |
| meningioma | 0.011 | FIG. 5 |
| notumor | 0.004 | FIG. 5 |
| glioma | 96.522 | FIG. 6 |
| meningioma | 1.912 | FIG. 6 |
| pituitary | 1.023 | FIG. 6 |
| notumor | 0.543 | FIG. 6 |
| glioma | 83.461 | FIG. 7 |
| pituitary | 13.031 | FIG. 7 |
| meningioma | 3.244 | FIG. 7 |
| notumor | 0.264 | FIG. 7 |

Table 1 presents the tumor predictions based on the described method for detecting Meningioma.

TABLE 2
PREDICTIONS FOR MENINGIOMA

| Label | Probability | Corresponding Figure |
|---|---|---|
| meningioma | 69.06 | FIG. 8 |
| notumor | 30.34 | FIG. 8 |
| glioma | 0.47 | FIG. 8 |
| pituitary | 0.13 | FIG. 8 |
| meningioma | 89.86 | FIG. 9 |
| notumor | 8.61 | FIG. 9 |
| glioma | 0.96 | FIG. 9 |
| pituitary | 0.58 | FIG. 9 |
| meningioma | 54.49 | FIG. 10 |
| notumor | 45.04 | FIG. 10 |
| glioma | 0.29 | FIG. 10 |
| pituitary | 0.18 | FIG. 10 |
| meningioma | 67.72 | FIG. 1 |
| pituitary | 17.35 | 11.jpg |
| notumor | 14.03 | 11.jpg |
| glioma | 0.90 | 11.jpg |
| meningioma | 92.48 | FIG. 12 |
| glioma | 4.82 | FIG. 12 |
| pituitary | 1.41 | FIG. 12 |
| notumor | 1.29 | FIG. 12 |

The Radiomics-Based Method for Predicting the Onset of Human Diseases Using Neural Networks and Color Space Analysis has a wide range of practical applications in medical diagnostics, particularly in enhancing early disease detection, improving personalized medicine, and supporting clinical decision-making. One of the primary applications is in the early detection of diseases such as cancer and neurological disorders. By analyzing medical images like CT, MRI, and PET scans using convolutional neural networks (CNNs), this method can extract radiomic features such as texture, shape, and intensity variations that may be too subtle for human detection. When combined with CIELAB color space transformations, these features are enhanced, allowing for earlier identification of conditions like tumors, Alzheimer's disease, and multiple sclerosis, where early intervention can significantly improve outcomes.

Another key application is in personalized medicine, where radiomics can predict how a patient will respond to specific treatments. In cancer therapy, for example, analyzing tumor radiomics data can predict responses to chemotherapy or radiation, enabling more tailored treatment plans. Additionally, the method can track disease progression over time, providing insights into whether a disease is advancing or responding to treatment, which is critical for chronic conditions like cancer or cardiovascular disease.

In terms of clinical decision support, the method can assist radiologists by automating the detection of regions of interest in medical images, providing machine learning-based predictions for disease onset, and generating detailed diagnostic reports. The integration of CIELAB color space transformations further enhances the visualization of tissue abnormalities, making it easier for clinicians to identify and interpret complex patterns in medical images. Heatmaps generated from these images guide clinicians to areas of concern for further evaluation, improving diagnostic accuracy.

The method's multi-modality compatibility also allows it to be used across various imaging techniques, such as CT, MRI, and PET scans, making it versatile for diagnosing a wide range of conditions. This adaptability ensures that the method can be applied in different medical contexts, providing comprehensive diagnostic insights across different diseases and imaging formats.

Additionally, the method significantly reduces the potential for human error by automating key parts of the diagnostic process. The CNN-based feature extraction process highlights patterns in grayscale images, and the color space transformation further enhances these features, leading to more accurate predictions. This automation is especially useful in large-scale clinical settings where radiologists are required to review a high volume of images quickly.

In the realm of remote diagnostics and telemedicine, this method can be deployed in remote healthcare settings, enabling healthcare professionals to analyze images from distant locations and provide timely diagnoses. This is particularly beneficial in rural or underserved areas where access to specialized radiologists is limited, making it a powerful tool for improving healthcare accessibility globally.

The google colab code to conduct the BrainTumor Predictions is also provided.

from google.colab import drive drive.mount(‘/content/drive’)
Mounted at /content/drive
from tensorflow import keras import numpy as np
import pandas as pd import os
# Load the Keras model
model = keras.models.load_model(‘/content/drive/MyDrive/CNN_Brain_model_V1.keras’)
# Function to preprocess the image
def preprocess_image(img_path, target_size):
img = keras.preprocessing.image.load_img(img_path, target_size=target_size) img_array =
keras.preprocessing.image.img_to_array(img)
img_array = np.expand_dims(img_array, axis=0)
img_array = img_array / 255.0 # Normalize the image (adjust if needed) return img_array
# Function to make predictions
def predict_image(model, img_array, top_k=5): predictions = model.predict(img_array)
probabilities = predictions[0] # Get the first array of predictions (for a single image) top_indices
= np.argsort(probabilities)[−top_k:][::−1] # Get the top K indices
return top_indices, probabilities[top_indices]
def save_to_excel(indices, probs, labels, image_path):
image_name = os.path.basename(image_path).split(‘.’)[0] # Get image name without extension
data = {‘Label’: [labels[i] for i in indices], ‘Probability’: probs*100}
df = pd.DataFrame(data)
# Save the file in the same directory as the image
excel_filename = os.path.join(os.path.dirname(image_path), f’{image_name}.xlsx’)
df.to_excel(excel_filename, index=False)
print(f“Predictions saved to {excel_filename}”)
# Function to process a directory and its subdirectories def process_directory(directory_path,
model, labels):
# Traverse the directory recursively
for root, dirs, files in os.walk(directory_path): for file in files:
if file.lower( ).endswith((‘.png’, ‘.jpg’, ‘.jpeg’)): # Filter image files image_path =
os.path.join(root, file)
print(f“Processing image: {image_path}”)
# Preprocess the image (change target_size based on your model's input size)
preprocessed_image = preprocess_image(image_path, target_size=(224, 224)) # Adjust as per
your model
# Make predictions
top_indices, top_probs = predict_image(model, preprocessed_image)
# Save the predictions to an Excel file with the image name save_to_excel(top_indices,
top_probs, labels, image_path)
if name == “main”:
# Path to the directory containing images
label_directory_path = ‘/content/drive/MyDrive/Brain Tumor MRI Dataset/Prediction’ #
Replace with your directory path
directory_path = ‘/content/drive/MyDrive/Brain Tumor MRI Dataset/Prediction’ # Replace
with your directory path # Check if the directory exists
if os.path.exists(directory_path):
# Assuming you have a list of labels corresponding to the model's output classes labels =
os.listdir(label_directory_path) # Replace with actual labels
# Process the directory process_directory(directory_path, model, labels)
else:
print(“Directory not found at the specified path.”)

The matlab code to developed the pseudo color and CIELABS based colorspace are also provided.

1. A computer-implemented method for predicting the onset of human diseases comprises several key steps. First, an original medical image in grayscale format is generated using diagnostic imaging techniques such as CT, MRI, or X-ray, facilitated by an image capture device. The image is then resized and cropped by a processor to isolate an area of interest for further analysis. In the feature extraction and preprocessing phase, the grayscale medical image is analyzed by a Convolutional Neural Network (CNN)-based feature extraction module to identify regions of interest based on variations in texture, shape, and intensity, providing critical data for early disease detection. The isolated area is further dissected into CIELAB color space values, optimized for human vision to enhance contrast and highlight subtle tissue variations. In the transformation and conversion stage, the identified grayscale regions of interest are converted into a pseudo-colored representation using a CIELAB-based color space transformation module, which enhances tissue contrast for improved clinical visualization. The pseudo-colored CIELAB image data is then compared with a predefined set of CIELAB matrices to detect specific tissue abnormalities and early disease markers. This pseudo-colored image is analyzed using a machine learning module, which predicts the likelihood of disease onset based on enhanced visual features derived from the CIELAB transformation. Finally, a machine learning classifier is used to generate actionable insights for clinical use, including a prediction of disease onset. An output report is created, which includes a heatmap highlighting areas of interest within the medical image, a probability score predicting the likelihood of disease onset, and diagnostic recommendations for use in medical diagnosis and treatment planning.

2. A computer-implemented system for predicting the onset of human diseases comprises several key modules working together to enhance clinical diagnosis. The Image Acquisition Module is configured to capture medical images in grayscale format using diagnostic imaging techniques such as CT, MRI, or X-ray. These images serve as the input data for the system's disease prediction process. The CNN-Based Feature Extraction Module analyzes these grayscale medical images and extracts important radiomic features, such as texture, shape, and intensity variations. It performs an initial down-selection to identify clinically relevant regions of interest that are crucial for the early detection of diseases. The Color Space Transformation Module converts the identified grayscale regions into a pseudo-colored representation using the CIELAB color space, which separates perceptual lightness (L*) from color components (a* for red-green and b* for blue-yellow). This transformation enhances tissue contrast, making subtle structures more visible to medical professionals. The Machine Learning Analysis Module analyzes these pseudo-colored images using machine learning algorithms, predicting the likelihood of disease onset by interpreting enhanced features based on patterns from labeled medical datasets. This module is designed to detect diseases such as cancer, cardiovascular conditions, and neurological disorders. Lastly, the Output Module generates a comprehensive report that includes a heatmap overlay highlighting areas of interest identified during the CNN and CIELAB analysis, a probability score predicting the likelihood of disease onset, and diagnostic recommendations for further clinical review or testing. This output is intended for use in clinical settings, assisting medical professionals in making informed decisions.

3. The method of claim 1, wherein the radiomic features extracted by the CNN include texture, shape, intensity variations, and spatial relationships within the grayscale medical images, and the CIELAB color space separates lightness (L*) from color components (a* for red-green and b* for blue-yellow), enhancing subtle variations in tissue structure for clinically improved disease detection; further comprising a machine learning classifier pre-trained using a labeled dataset of medical images with known disease outcomes to refine its predictive capability in a clinical diagnostic setting; wherein the output report includes a detailed description of the likelihood of disease onset based on a probability score generated by the machine learning classifier, and the medical images used in the method are sourced from multiple imaging modalities including CT, MRI, and X-ray, with an initial image preprocessing step that enhances contrast and optimizes the grayscale medical images for CNN feature extraction, enabling more accurate and actionable clinical decisions.

4. The system of claim 2, wherein the CNN-based feature extraction module is configured to automatically identify texture, shape, and intensity features indicative of disease presence, and the color space transformation module applies the CIELAB color space to enhance perceptual lightness (L*) and distinguish subtle tissue variations using color channels a* and b*; wherein the machine learning module is configured to use a pre-trained algorithm to classify the likelihood of disease based on the enhanced features from the pseudo-colored images in a manner that supports clinical diagnosis; further configured to generate a heatmap highlighting the areas of the medical image that were most influential in the disease prediction using both CNN and CIELAB analysis; wherein the image acquisition module is compatible with multiple imaging modalities, including CT, MRI, and X-ray, allowing for analysis of different medical imaging types for improved diagnostic versatility; and wherein the machine learning module provides interpretability features such as saliency maps or layer-wise relevance propagation to explain the predictions made by the system, ensuring that healthcare professionals can trust and act upon the system's recommendations.
